(19) United States
(12) Patent Application Publication
(10) Pub. No.: US 2007/0191687 A1
(43) Pub. Date: Aug. 16, 2007

(54) Diagnostic tool for pulmonary diseases

(76) Inventor: Claus JUSTUS, Welgesheim (DE)

Correspondence Address: STERNE, KESSLER, GOLDSTEIN & FOX P.L.L.C., 1100 NEW YORK AVENUE, N.W., WASHINGTON, DC, 20005, US

(21) Appl. No.: 10/584,915

(22) PCT Filed: Dec. 23, 2004
(86) PCT No.: PCT/EP04/14689

§ 371 Date: Oct. 27, 2006

(30) Foreign Application Priority Data
Dec. 29, 2003 (EP) 03029952.3

(51) Int. Cl. A61B 5/00; A61B 5/08

(52) U.S. Cl. 600/300; 128/920; 600/529; 600/301

(57) ABSTRACT

A diagnostic tool for pulmonary diseases comprises a display unit (2) for displaying predefined diagnostic questions relating to the pulmonary disease and for outputting a diagnostic prognosis on the disease, an input unit (4) for receiving responses from a user to the diagnostic questions displayed on the display unit (4), a storage unit (8) for storing the predefined questions and the interactively input responses, a calculation unit (6) for assigning each received response a predetermined count value, adding up the count values obtaining a final count value, assigning the final count value the diagnostic prognosis using a pre-defined result table (15) stored in the storage unit (8). The diagnostic tool according to the present invention allows the general practitioner or the patient to carry out a fast first diagnosis on functional lunge disease, in particular COPD. If a high probability, for example in percentage points, is established, further, more detailed diagnostic steps may be undertaken.

The present invention relates to a diagnostic tool for the diagnosis of pulmonary diseases like chronic obstructive pulmonary disease (COPD).

### DESCRIPTION OF RELATED ART

Diagnosis and therapy of pulmonary diseases is based on published medical guidelines like for example of the American Thoracic Society, Standards for the Diagnosis and Care of Patients with Chronic Obstructive Pulmonary disease, Am I Respir/Crit Care Med. Vol. 52, pages S77-S120, 1995, Siafakas, N. M., Vermeire, P., Pride, N. B., ERS Consensus Statement: Optimal Assessment and Management of Chronic Obstructive Pulmonary Disease (COPD), Eur Respir J, 8, 1398-1420, 1995; National Heart, Lung and Blood Institute, 1995. Global Initiative for Asthma. U.S. Government Printing Office, Washington, DC. Publication No. (NIH) 95-3659. Based on the patient's disease history, patient's symptoms and measurement results like spirometry/lung function measurements using a spirometer the physician makes his/her diagnosis, discusses the same with the patient and enters it into the patient's records. Depending on the diagnosis the physician checks whether or not a medication is appropriate and when the patient is already on a treatment regimen proposes, if necessary, alternative therapies. The success of the therapy is then reviewed during the next visits of the patient.

For making his/her diagnosis, the physician has to review the patient records and, if respiratory measurements are carried out and have to be interpreted, he/she frequently has to look up in detail the interpretation of the measurement values in the relevant guidelines as well as the implication of these results with regard to chronic obstructive lung disease. This process is complicated and time-consuming.

Epidemiological data from the national health interview survey and the third national health and nutrition and examination survey in the United States illustrate relationships in the prevalence of asthma and COPD in nationally representative samples. Asthma prevalence in adults declines in weight from 5 to 10% at age 20 to 40 to 4 to 8% above age 60. COPD is uncommon in adults under age 40 but steadily increases with age, surpassing asthma in older adults. These findings suggest that asthma screening is most useful in adults up to approximately age 40, after which COPD screening and differential diagnoses are of comparable or greater utility.

A system for assisting in the diagnosis of functional lung diseases based on spirometric measurement data is known from EP 1271384 A1.

### SUMMARY OF THE INVENTION

It is therefore an object of the present invention to provide a physician, case manager or the patient with a diagnostic tool allowing to give a first, fast diagnostic prognosis of pulmonary diseases, in particular COPD at low cost.

The present invention provides a diagnostic tool adapted to assist in the diagnosis of pulmonary diseases based on data not including lung function measurement data comprising a display unit for displaying predefined questions relating to the pulmonary disease, and for outputting a diagnostic prognosis on the disease, an input unit for receiving responses from a user to the questions displayed on the display unit, a storage unit for storing the predefined questions and the interactively input responses, a calculation unit for assigning each received response a predetermined count value, adding up the count values obtaining a final count value, assigning the final count value the diagnostic prognosis using a predefined result table stored in the storage unit.

The diagnostic tool according to the present invention allows the general practitioner or the patient to carry out a fast first diagnosis on functional lunge disease, in particular COPD. If a high probability, for example in percentage points, is established, further, more detailed diagnostic steps may be undertaken.

The diagnostic questions preferably comprise questions about patient demographic data, smoking status and subjective disease symptoms of a patient. The demographic data may include the age, sex and/or the body mass index (BMI) of the patient. The smoking status may include the current smoking status (smoker/non-smoker) and the smoking history. The latter can preferably be taken into account by a transformation table in matrix form assigning combinations of the average smoking intensity (cigarettes per day) and the smoking duration (in years a respective count value) respective count values.

The subjective disease symptoms may include the occurrence of short breath, phlegm and chest wheezing or whistling.

The diagnostic tool may be formed as an electronic or a mechanic instrument. The electronic instrument can preferably be implemented as a handheld device or be integrated into a laptop computer or organiser. The diagnostic tool may comprise a scroll wheel and an operation button allowing one-hand operation of the diagnostic tool.

The diagnostic tool may be operated in remote application, as for example by Intemt, Email, SMS or MMS.

The diagnostic tool may be used for diagnosing previously undiagnosed persons, as a tool for the recruitment of participants for clinical trials and as a differential diagnoses tool allowing to differentiate COPD from other chronic obstructive respiratory diseases such as asthma.

The present invention and further features, objects and advantages thereof will become more readily apparent from the following detailed description of particular embodiments thereof, taken in conjunction with the accompanying drawings.

### BRIEF DESCRIPTION OF THE DRAWINGS

FIG. 1 schematically illustrates a first embodiment of the diagnostic tool according to the present invention.

FIG. 2 schematically illustrates the functional units of an embodiment of the diagnostic tool according to the present invention.

FIG. 3 illustrates a second embodiment of the diagnostic tool according to the present invention.

FIG. 4 illustrates a third embodiment of the diagnostic tool according to the present invention.

FIG. 5 shows an example of a transformation table for use with the diagnostic tool according to the present invention.

FIG. 6 shows an example of a result table for use with the diagnostic tool according to the present invention.

### DETAILED DESCRIPTION OF PREFERRED EMBODIMENTS

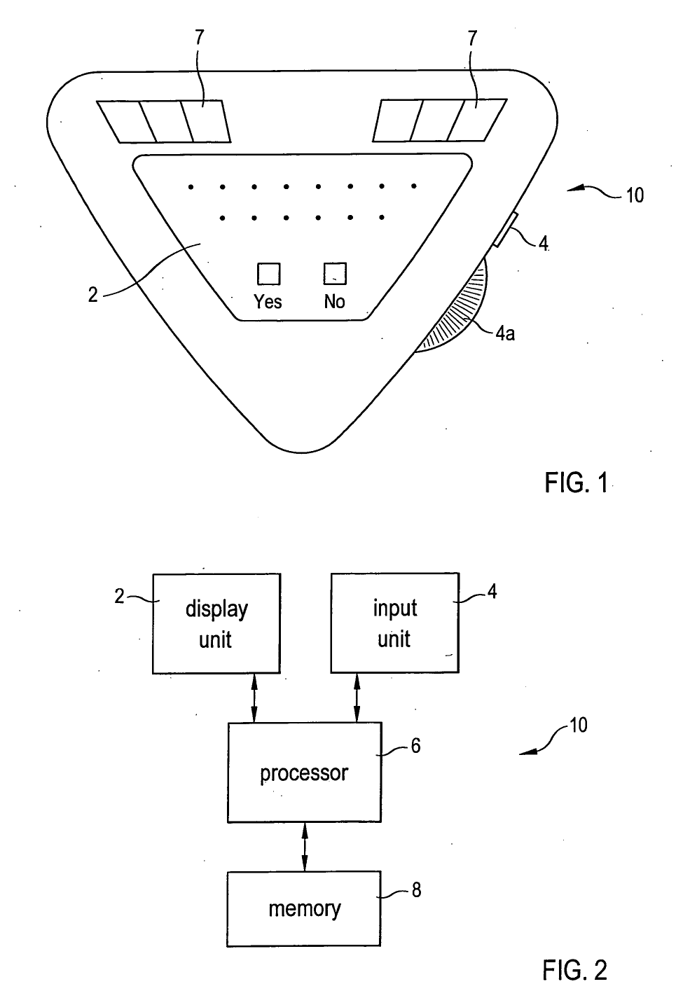

FIG. 1 illustrates a first embodiment of the diagnostic tool according to the present invention. The diagnostic tool 10 comprises a housing preferably made of synthetic resin or any other suitable material. The device comprises a display 2 as for example a liquid crystal display (LCD) and manual input means 4, 4a. After starting operation of the diagnostic tool pre-defined questions significant for the probability of the occurrence of the disease like COPD as for example the question “Are you currently smoking?” are displayed on the display means 2. The selection of the diagnostic questions will be discussed in more detail later. The user, a physician, case manager or patient, can then respond to the question by moving a cursor between a “yes” and a “no” field by means of a scrolling wheel 4a and confirming the respective response by pressing input-or enter-button 4. The arrangement of button 4 and scrolling wheel 4a allows a single-handed operation of the diagnostic tool 10. In order to avoid the necessity of battery replacement photovoltaic cells 7 and a suitable capacitor (not shown) are provided as power source.

When the user has responded to a diagnostic question by pressing button 4 the next question is shown on display 2. The user then again inputs his response until an answer to the last question has been given by the user. The final result is then calculated as will be explained in more detail later and displayed on a display device 2.

Instead of yes-or no-questions it is also possible for a user to choose among a plurality of different responses by navigating between different response fields using the scroll wheel 4a. On the question “What is your age?”, the user could for example choose between response fields “under 40”, “40 to 49”, “50 to 59”, “60 to 69”, “70 or older”.

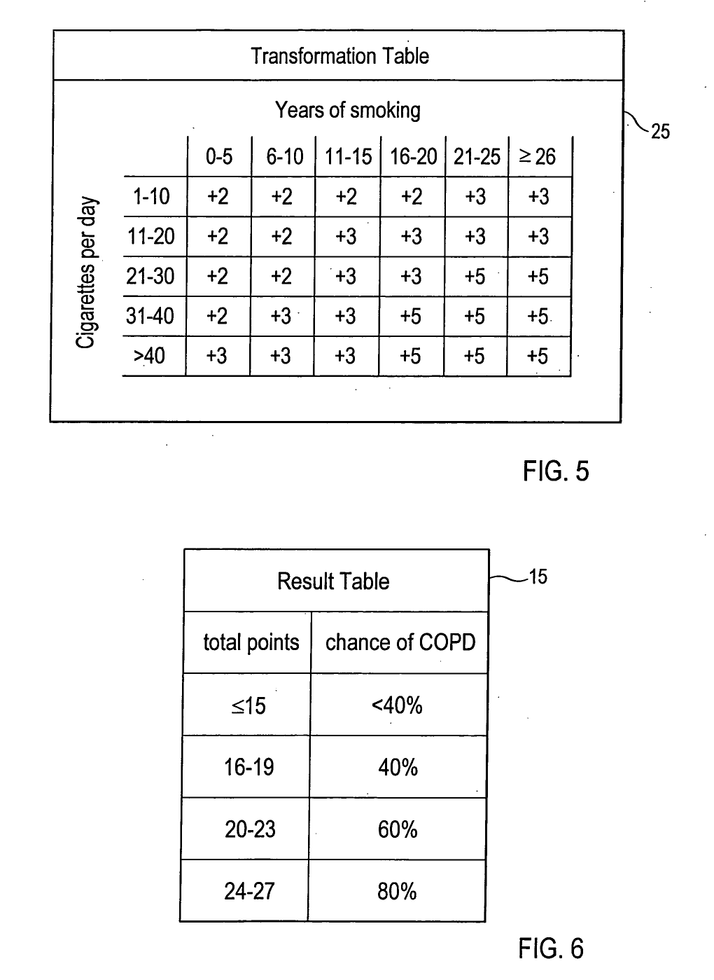

The functional units of the diagnostic tool 10 are shown in the block diagram of FIG. 2. A processor 6 controls the operation of display unit 2 and input unit 4, consisting e.g. of button and scroll wheel or any other suitable input device. In addition, processor 6, which may be a well-known microprocessor device, controls memory 8 on which the predefined questions and the predefined tables as the transformation table shown in FIG. 5 and the result table of FIG. 6 are stored in a non-volatile memory section and the interactively given responses and the current count value are stored in a dynamic memory section. Moreover, the processor 6 performs the calculation of the count value and the assignment of the diagnostic prognosis based on the final count value.

The operation of the diagnostic tool 10 according to the present invention is as follows:

The user starts operation by pressing the input button 4 or a separate start key. Processor 6 then controls the display unit 2 to display the first question like e.g. “What is your age?”. The user then gives his response by navigating a cursor by navigation wheel 4 to the corresponding response field and confirming the input by pressing confirmation button 4. Then, on the display 2 the next question is shown, for example a yes/no-question like “Are you currently smoking?”. The user then has to choose either the “yes” or “no” response symbol. The next question may be about the smoking history of the patient. This question consists of two parts, namely the average smoking intensity in cigarettes per day and the total smoking time in years. After the user has input the corresponding responses by “clicking” on the corresponding response fields the count value corresponding to the smoking history is calculated by processor 6 using a transformation table as shown in FIG. 5. In vertical direction the average smoking intensity and in horizontal direction the total smoking time is given. The count value corresponding to the smoking history of a patient can then be taken from the respective element of the table shown in FIG. 5.

Typically some questions about subjective disease symptoms of the patients follow including being short of breath, occurrence and quantity of phlegm, and/or the occurrence of chest wheezing or whistling. To each response a corresponding count value is assigned, which count value may also be a negative value. Processor 6 performs a summation of all count values and calculates a final count value as result when the response to the last question has been input by the user. The final count value is then translated into a diagnostic prediction using a result table an example of which is shown in FIG. 6. A final count value of 16 to 19 for example corresponds to a COPD probability of 40% and a count value of between 24 and 27 a COPD probability of about 80%. High percentage values suggest further, more detailed a diagnosis of the patient.

The selection of the diagnostic questions, the transformation of the responses into account values and the diagnostic prognosis values of the result table are the result of extensive clinical studies. In these studies the predictive ability and statistical significance of a plurality of selected question items have been investigated.

For identifying COPD (case finding) the following questions showed statistically significant relation with spirometric results (post-bronchodilator FEV1/FVC<0,70). In a study the following questions achieved sensitivities of 58.7%-80.4% and specificities of 57.5%-77.0%, depending on the scoring system used. Predictive values range from 30.3%-37.0% for a positive test and 89.0%-92.7% for a negative test.
Age group (in years);
Pack-years smoked (“How many cigarettes do you currently smoke each day [if you are an ex-smoker, how many did you smoke each day]?” and “What is the total number of years you have smoked cigarettes?”);
Body mass index (calculated from height and weight);
Weather affects cough (“Does the weather affect your cough?”);
Phlegm without a cold (“Do you ever cough up phlegm [sputum] from your chest when you don't have a cold?”);
Phlegm in the morning (“Do you usually cough up phlegm [sputum] from your chest first thing in the morning?”);
Wheeze frequency (“How frequently do you wheeze?); and
Have had any allergies (“Do you have or have you had any allergies?”).

For differentiating COPD and asthma a similiar study found the following 9 questions showing statistically significant relationships with obstruction. This candidate questionnaire achieves sensitivities of 53.8%-83.3% and specificities of 58.8%-88.2%, depending on the scoring system used. Predictive values range from 60.7%-77.8% for a positive test and 71.4%-82.2% for a negative test:
Age group (in years);
Pack-years smoked (“How many cigarettes do you currently smoke each day [if you are an ex-smoker, how many did you smoke each day]?” and “What is the total number of years you have smoked cigarettes?”);
Recent cough (“Have you coughed more in the past few years?”);
Breathing-related work loss (“During the past 3 years have you had any breathing problems that have kept you off work, indoors, at home, or in bed?”);
Hospitalization for breathing (“Have you ever been admitted to hospital with breathing problems?”);
Recent breathlessness (“Have you been short of breath more often in the past few years?”);
Quantity of phlegm (“On average, how much phlegm [sputum] do you cough up most days?”);
Cold usually goes to chest (“If you get a cold, does it usually go to your chest?”); and

The resulttable may preferably based on a scoring system using two cut-points intended to place the persons within one of three zones:
Persons with a high likelyhood of having obstruction (high pridictive value of a positive test; red zone),
persons with a low likelyhood of obstruction (high priductive value of a negative test; green zone) and
an intermediate zone (yellow zone).

Based on the above-mentioned questions and the 3-zone scoring system an example of a questionnaire and the corresponding result table (scoring) for case-finding as well as a differential diagnosis against asthma is given in the following table 1.

TABLE 1

| | | Scoring | | | |
|---|---|---|---|---|---|
| | | Case- | | Differential | |
| | | Finding | | Diagnosis | |
| What is your age? | 40-49 | 0 | points | 0 | points |
| | 50-59 | 17 | points | 16 | points |
| | 60-69 | 33 | points | 22 | points |
| | 70 or older | 38 | points | 25 | points |
| Packyears Smoked | 0-14 | 0 | points | 0 | points |
| | 15-24 | 14 | points | 12 | points |
| | 25-49 | 17 | points | 19 | points |
| | 50 or over | 24 | points | 21 | points |
| BMI Thirds (will refer to nomograph) | Lowest (<18 male, <19 | 0 | points | | |
| | female) | | | | |
| | Middle (18-28 male, 19- | −20 | points | | |
| | 29 female) | | | | |
| | Highest (>28 male, >29 | −27 | points | | |
| | female) | | | | |
| Do you ever cough up phlegm (sputum) from | YES | 20 | points | | |
| your chest when you don't have a cold? | NO | 0 | points | | |
| (phlegm1) | | | | | |
| Do you usually cough up phlegm (sputum) | YES | −21 | points | | |
| from your chest first thing in the morning? | NO | 0 | points | | |
| (phlegm2) | | | | | |
| How frequently do you wheeze? (wheeze4) | Never | 0 | points | | |
| “Alternate: Do you ever wheeze? Yes-No” | Occasionally/1/wk/ | 20 | points | | |
| | Every day | | | | |
| Do you have or have you had any allergies? | YES | −22 | points | | |
| (priorhx7) | NO | 0 | points | | |
| On average, how much phlegm (sputum) do | None/Less than 1 | | | 0 | points |
| you cough up most days? (phlegm4) | tablespoon/day | | | | |
| “Alternate: Do you cough up more than 1 tablespoon of phlegm | 1 tablespoons/day | | | 17 | points |
| (sputum) on most days? Yes-No” | | | | | |
| During the past 3 years have you had any | YES | | | −14 | points |
| breathing problems that have kept you off | NO | | | 0 | points |
| work, indoors, at home, or in bed? (dyspnea1) | | | | | |
| Have you ever been admitted to hospital with | YES | | | 18 | points |
| breathing problems? (dyspnea2) | NO | | | 0 | points |
| Are you taking any treatment to help your | YES | | | 19 | points |
| breathing? (priorhx9) | NO | | | 0 | points |

| | |
|---|---|
| TOTAL | TOTAL |
| 55 | 60 |
| 21-54 | 36-59 |
| 20 | 35 |

A final count value of more than 55 (60) corresponds to the red zone, values between 21 and 54 (36 and 59) to the yellow zone and final count values below 20 (35) correspond to the green zone.

According to the present invention the functions of identification of COPD and differntial diagnosis are preferably integrated within one tool.

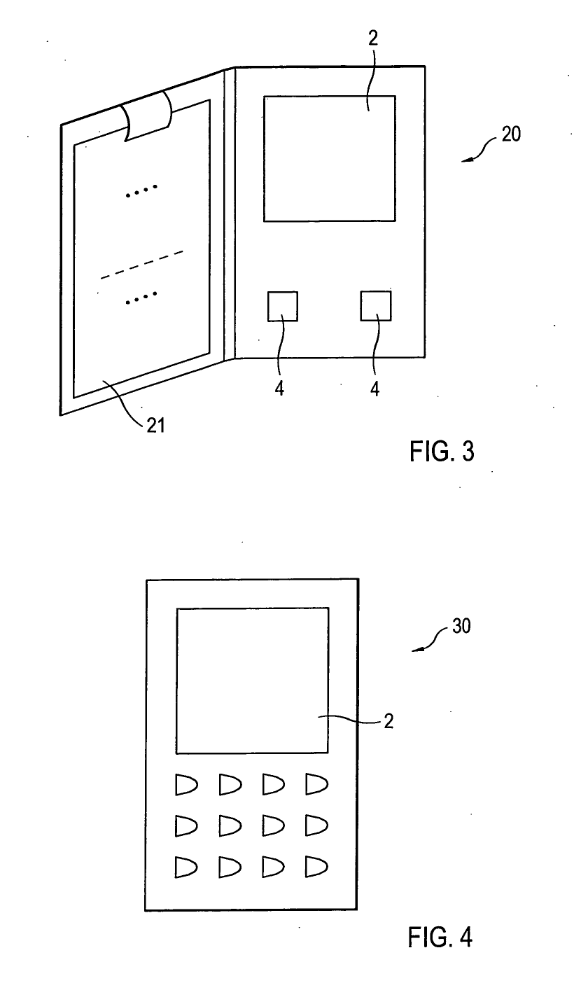

Further embodiments of the diagnostic tool according to the present invention are shown in FIGS. 3 and 4. FIG. 3 illustrates a diagnostic tool 20 comprising a casing incorporating the display, processor and input units of the device as well as means (21) for holding a prescription pad and possibly a pen for the physician. As shown in FIG. 3, diagnostic tool 20 may be operated by two input units 4, one for navigation and one for input confirmation similarly to the input device of a laptop computer. Alternatively, the functionality of the diagnostic tool my be incorporated in a laptop computer, a smart phone or organiser 30 as shown in FIG. 4. As a further alternative, the diagnostic tool according to the invention may be embodied as a mechanical device.

1. A diagnostic tool adapted to assist in the diagnosis of pulmonary diseases, based on data not including lung function measurement data, comprising:
a display unit (2) for displaying predefined diagnostic questions relating to the pulmonary disease, and for outputting a diagnostic prognosis on the disease,
input unit (4) adapted to receive responses from a user to the diagnostic questions displayed on the display unit (2),
a storage unit (8) having stored thereon the predefined questions and the interactively input responses, and
a calculation unit (6) adapted to:
assign each received response a predetermined count value,
add up the count values obtaining a final count value, and
assign the final count value the diagnostic prognosis using a predefined result table (15) stored in the storage unit (8).

2. The diagnostic tool of claim 1, wherein the diagnostic prognosis is given as a percentage value for the general practitioner or a risk factor for the patient.

3. The diagnostic tool of claims claim 1, wherein the diagnostic questions comprise questions about patient demographic data, smoking status and subjective patient disease symptoms.

4. The diagnostic tool of claim 3, wherein the demographic data include age, sex and/or body mass index (BMI) of the patient.

5. The diagnostic tool of claim 3, wherein the questions about the smoking status of the patient include questions about current smoking status and aggregate smoking history.

6. The diagnostic tool of claim 5, wherein the calculation unit (6) utilises a transformation table (25) assigning predetermined count values to different combinations of smoking intensity (cigarettes per day) and smoking duration (in years).

7. The diagnostic tool of one of claim 3, wherein the subjective patient disease symptoms include breathing restrictions, phlegm and chest wheezing or whistling.

8. The diagnostic tool of one of claim 1, wherein the diagnostic tool is formed as an electronic instrument (10).

9. The diagnostic tool of claim 8, being formed as a handheld device comprising an input key (4) and a scroll wheel (4a) allowing one hand operation of the diagnostic tool.

10. The diagnostic tool of claim 8, comprising photovoltaic cells (7) as power source.

11. The diagnostic tool of one of claim 8, wherein the diagnostic tool is integrated with a handheld computer or organiser.

12. The diagnostic tool of one of claim 8, wherein the diagnostic tool comprises a casing for housing a prescription pad (21) and a pen.

13. The diagnostic tool of one of claim 1, being formed as a mechanical device.

14. The diagnostic tool of claim 1 which can be operated in remote application, as for example by Internet, by Email, SMS or MMS.

15. The diagnostic tool of claim 1, wherein the diagnostic tool is used for diagnosing chronic obstructive pulmonary disease (COPD).

16. The diagnostic tool of claim 1, wherein the diagnostic tool is used for diagnosing previously undiagnosed persons.

17. The diagnostic tool of claim 1, wherein the diagnostic tool is used as a tool for the recruitment of participants for clinical trials.

18. The diagnostic tool of claim 1, wherein the diagnostic tool is used as a differential diagnosis tool allowing to differentiate COPD from other chronic obstructive respiratory diseases such as asthma.

19. A method of diagnosing chronic obstructive pulmonary disease (COPD) in a person, wherein the method comprises the input and evaluation of responses from said person with the aid of a diagnostic tool of claim 1.

20. A method of diagnosing COPD in a previously undiagnosed person, wherein the method comprises the input and evaluation of responses from said person with the aid of a diagnostic tool of claim 1.

21. A method for the recruitment of participants for clinical trials, wherein the method comprises the input and evaluation of responses from potential participants with the aid of a diagnostic tool of claim 1.

22. A method to differentiate COPD from other chronic obstructive respiratory diseases, wherein the method comprises the input and evaluation of responses from a person with the aid of a diagnostic tool of claim 1.

23. A method to differentiate COPD from asthma, wherein the method comprises the input and evaluation of responses from a person with the aid of a diagnostic tool of claim 1.
